(19) United States
(12) Patent Application Publication
(10) Pub. No.: US 2019/0231595 A1
(43) Pub. Date: Aug. 1, 2019

(54) ERGONOMIC SLEEP MASK

(71) Applicant: The Art of Alchemy, Edgewater, NJ (US)

(72) Inventors: Catherine S. Holtz, Edgewater, NJ (US); Elizabeth A. Daily, Edgewater, NJ (US)

(21) Appl. No.: 16/262,780

(22) Filed: Jan. 30, 2019

Related U.S. Application Data
(60) Provisional application No. 62/623,567, filed on Jan. 30, 2018.

(51) Int. Cl. A61F 9/04; A61K 36/886; A61K 31/355

(52) U.S. Cl. CPC A61F 9/04; A61K 31/355; A61K 36/886

(57) ABSTRACT

The present invention is a sleep mask with many unique features including tension reduction from the securing bands, a light blocking pillow, ergonomic/pre-curved shaping, an eye space, ear comfort features, slip over comfort which can account for various hair styles and various head positions of the wearer, and therapeutic, beauty and relaxing features. The overall experience for the wearer is an extraordinary fit due to data and design.

### COPYRIGHT NOTICE

A portion of the disclosure of this patent document contains material that is Subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### FIELD OF THE INVENTION

One or more embodiments of the invention generally relate to sleep masks. More particularly, the invention relates to an ergonomically designed sleep mask.

### BACKGROUND OF THE INVENTION

There are a number of situations where people find themselves seeking a restful sleep often in compromised circumstances. Examples would include routine sleeping at home, travel by air, car or train, sleeping in hotels, unfamiliar surroundings or environments with significant ambient light, daytime sleeping or sleeping while another reads or watches television. Prior art in this area differs in the problems it sets out to solve, seeking to address other issues affecting sleep for example: sound, movement, stress and sleeping while seated, or the method it employs to solve similar problems. Limitations of prior art are often associated with poor fit, creating tightness and pressure on the head; unsightly hair dents; tangle or damage to hair and clothing; ear rubbing; strap slipping or pressure on the eyes restricting blink-ability and disturbing contacts or eye makeup. Many available masks combine a complex array of functions or have found distinctly different solutions to one or other of the issues identified, at the cost of an undesirable appearance. Thickness and malleability can be particular issues for side sleepers and masks found in the prior art can be rendered ineffective if shifted during use. The present invention comprises a number of unique aspects which deal with these problems and can be clearly differentiated from prior art.

Although prior art attempts to address face/head tension, for example U.S. Design Pat. No. D388,812, none do so in the same way or so effectively.

Related prior art often display lower edge light blocking pillows, for example U.S. Pat. Nos. 1,924,315, 2,305,080, and 2,191,937. These uniform pillows are made of a tube centered around the nose and don't have the requisite depth to fill in the voids either side of the nose.

U.S. Pat. Application 2014/0041091 discloses prior art which is designed to be worn sitting down with the wearers head secured to a seat back. This mask does include a malleable light blocking ‘nose bridge’, although it is more firm in construction than the present design and covers a shorter span of the lower edge of the mask (ends before temples). Though this nose bridge might fit perfectly in place while sitting upright, or lying prone, as soon as the wearer shifts to the side pressure would be applied to the side of the mask causing shifting and the firmer nose bridge to be pushed away from the wearer's face allowing light to enter. In addition, the more rigid areas press against parts of the nose, causing discomfort. US Pat. Application 2017/0020767 (U.S. Design Pat. No. D777,824) does contain a nose bridge element but this operates very differently, delivering acupressure/reflexology to the users face.

Prior art does not seem to have been designed to curve to the face. Flat masks such as U.S. Pat. Nos. 1,924,315 and 2,305,080 would bunch up in an effort to shorten the interior surface of the mask, when curved over a wearer's face. In the areas where the fabric is bunched up or creased, light may seep in, because it's no longer a smooth, taut surface with full-face contact. This, in combination with the lower light blocking pillow, and then the deformation of the pillow padding for different shapes, is what ensures light blocking and comfortable fit.

Another area where the current invention is unique is the approach taken to free space around the eye area or ‘blink-space’. U.S. Pat. Nos. 2,191,937 and 2,305,080 and 2,671,898 are early attempts to address the issue of blink-space by using padded pillows at the lower and upper edges to hold the mask off the wearer's face. Similarly, U.S. Pat. Application 2010/0122398 (which primary purpose is to protect the wearer's eyelashes during sleep) uses a fairly rigid perimeter portion which contacts the wearer's face creating a space between the inner surface of the mask and the face. U.S. Pat. No. 4,872,217 and U.S. Pat. Application 2014/0041091 (cited above) address the issue of blink-space by including a pair of eye depressions, whereby the person's eyelids and eyelashes do not contact the outer layer. Similarly, U.S. Pat. Application 2016/0008175 includes eye cavities. The depressions/cavities/perimeters cited in the foregoing prior art need to be accommodated by extra padding, delivering high volume masks. Another approach to accommodating blink space is to create an eye area which visibly bows outward, so that the mask appears convex on the outside in the area of the eyes creating “an alien effect”: for example, U.S. Pat. Application 2014/0331383. Because the current invention is specifically to be worn in public, an adverse “alien” effect is very undesirable.

Prior art addresses mask securing in a wide variety of ways. For example there are sleep masks which are secured to the ears U.S. Pat. Nos. 1,047,163 and 2,243,982, this must be uncomfortable, and others fasten using Velcro®, snaps or buttons, for example U.S. Pat. Application 2016/0008175 and U.S. Design Pat. No. D489,749, which can tangle or damage hair or clothing or hurt the wearer's head during sleep.

U.S. Pat. No. 9,168,177 (also published as U.S. Pat. Application 2013/0117899) and U.S. Pat. Application 2016/0295946 are slipover type sleep masks but differ in overall construction. Both have broad, substantial, tubular-shaped bodies which slip over and encircle the head, like a sleeve. These devices do not claim a nose pillow or therapeutic aspect, are large on the head, possibly inducing claustrophobia and presumably very hot to wear.

Prior art discloses sleep masks which include devices intended to secure the head of the wearer, disabling movement when travelling in a seated position: For example, U.S. Pat. No. 1,924,315, U.S. Pat. Application 2013/0152947, U.S. Pat. Application 2014/0041091 and U.S. Pat. No. 9,186,276. These devices have drawbacks such as are complicated to use, unsightly, often bulky to carry and do not adapt to prone sleeping.

U.S. Pat. No. 8,852,073 comprises audible and visual components to promote a state of relaxation.

Prior art discloses devices which include both a sleep mask and pillow element. For example, U.S. Pat. Application 2016/0022045 and U.S. Pat. No. 8,239,987 which include neck cushion devices intended for use in a seated position or U.S. Pat. Application 2012/0210516 which includes neck and ear pillows also designed for seated use. U.S. Pat. Application 2016/0000607 discloses a sleep mask pillow combination, designed for use in a prone position. These devices either address sleeping prone or in a seated position and are not easily adaptable between the two, they are not compact for easy carrying.

Prior art equipment also exists to minimize sound sensed by a sleeping individual as well as a mask element. For example, U.S. Pat. Nos. 2,942,270, 2,537,768, 5,343,561, U.S. Pat. Application 2016/0346129, U.S. Design Pat. No. D485,854 and U.S. Design Pat. No. D465,234. The addition of sound reducing equipment adds significantly to volume and diminishes simplicity and ease of transport.

Prior art also discloses sleep masks which claim to be light blocking but have the primary purpose of providing some therapeutic benefit. For example, U.S. Pat. Application 2016/0008175 claims to block out light but is primarily a gel pad that provides refreshing relief to the face and eyes of the wearer. Similarly, U.S. Pat. No. 6,537,308 and U.S. Pat. Application 2015/0173487 have the primary purpose of administering beauty care products. U.S. Pat. No. 6,293,900 delivers magneto therapy.

The foregoing prior art differ in the issues they address and where an element of prior art addresses a problem solved by the present invention, the product and method used is entirely different and less effective.

### SUMMARY

In view of the disadvantages described in prior art, the present invention provides a new ergonomically designed sleep mask device which comprises: a light blocking eye shield, including a soft contoured pillow to block light at the bottom edge of the mask, covering: the nose bridge; upper nasolabial area; cheekbones and then tapering off towards the temples, which conforms to the hollows of the face on either side of the nose and creates a touch free eye zone; a continuous dual slip over strap attached by connectors which distribute tension away from the face and back of the head; and is adjustable by sliders. The mask is conveniently light in weight and low in volume. The sleep mask fabric is hypoallergenic and can be infused with aloe and Vitamin E.

It is therefore the principal object of the present invention to provide an improved low volume sleep mask which combines efficacy in the areas of light blocking and comfort while delivering an appealing, understated appearance suitable for wearing in public.

A further object is to provide a mask which will rest comfortably on the bridge of the nose and on the adjacent portions of the face, without creating tension.

A further object of the present invention is to block 100% or as close to 100% as possible, like 95%, 96%, 97%, 98% and 99% of ambient light from the user's eyes.

Another object of the present invention is to provide a sleep mask that specifically fits the face well.

Another object of the invention is to provide a sleep mask which can easily be placed in position and will remain where placed without becoming accidentally dislodged under ordinary conditions, while worn in either seated or prone positions.

Another object of the present invention is to provide a sleep mask which includes a touch free eye zone.

Another object of the present invention is to provide a sleep mask with an easy to adjust continuous elastic slip-on securing strap with no fasteners to tangle or damage hair or clothing or hurt the wearer's head during sleep.

A further object is to provide a sleep mask which offers a wide range of adjustability for use by persons of different sizes.

Another object of the present invention is to provide a method for enhancing the sleep experience while delivering a subtle therapeutic effect.

A further object of the present invention is to provide a sleep mask with a securing band which rests clear of the wearers' ears.

Another object of the present invention is to provide a sleep mask which can be readily washed as needed.

### BRIEF DESCRIPTION OF THE DRAWINGS

The novel features believed characteristic of the current invention are set forth in the appended claims. The invention itself however, will best be understood by reference to the following detailed description of an illustrative embodiment and the appended drawings in which:

FIG. 1 is a front view of a sleep-mask device, illustrated as worn by a person, in accordance with an embodiment of the invention;

FIG. 2 is a rear view of the device with the attaching bands omitted;

FIG. 3 is a cross-sectional view of a sleep-mask device taken along line 3 of FIG. 1, illustrated as worn by a person, in position over the eyes, showing the positioning of the light blocking pillow;

FIG. 4 is a right side and partial cross-sectional view of the invention;

FIG. 5 is a rear view of a sleep-mask device with the attaching bands;

FIG. 6 is a close-up view of the right side of the device showing the assembly of the connectors and securing band;

FIG. 7 is a bottom perspective view of the sleep mask device, illustrated as worn by a person, in accordance with an embodiment of the invention; and

FIG. 8 is a bottom perspective view of the sleep mask showing how the mask is pre-curved to fit the face.

### DETAILED DESCRIPTION OF THE PREFERRED EMBODIMENT

There are several unique features of the present invention include tension reducing from the securing bands, the light blocking pillow, the ergonomic, pre-curved shaping, sufficient eye space, the ear comfort of the wearer, the slip over comfort accounting for various hair styles and various head positions of the wearer, and the therapeutic and relaxing features.

At least one unique feature of the current invention is the approach taken to reducing tension on the face and back of the head. This is achieved by the interplay of the side connectors; a continuous, anti-tension elastic securing band and at least one or two layers of batting over the face. Batting is considered to be sheets of fibers. The anti-tension securing band connectors are made from corded ribbon which forms a “V” shape which distributes tension by following the top and bottom lines of the mask. The “V” shape ribbon is connected to the continuous elastic band. The continuous elastic securing band then forms two straps, creating unbroken lines from the connectors that continue to distribute tension from the top and bottom of the mask and create a pressure free eye zone. The involvement of two securing bands lessens the tension on any one line, distributing it evenly over the two lines of elastic, to lessen the amount of pressure on the face and back of head, while creating a firm even tension on the face. The nature of the sliders allows for micro-adjustment of tension and location. The preferred embodiment utilizes soft-stretch elastic which stretches following the application of only a small degree of force. This type of elastic exerts far less pressure than an elastic which requires a high degree of force to activate the stretch.

Also the thickness of two layers of facial batting, for example, has been carefully adjusted to ensure that facial pressure minimized. The number of batting layers is not limited to two layers. Other embodiments of the invention include one layer of batting as well as a plurality of layers of batting separated either by differences in material densities or by thin fabric. The plurality of layers of batting include 2, 3, 4, 5, 6, 7, 8, 9 and 10 layers. In even further embodiments, no batting is used or needed as the fabric for the sleep mask itself is such that the cushioning effect of the batting and feel of the mask is supplied by the fabric alone. In such an embodiment without batting, thick fabric for the sleep mask is used and could contain the other features of the sleep mask, such as darting, as described herein. Various fabrics of various thicknesses can be chosen for the sleep mask. Some fabrics, if used, would require batting between an inner layer and an outer layer, while other fabrics could be used alone without additional batting as long as the shape and feel of the mask is achieved, as described herein.

The approach taken to light blocking is also unique. The current invention employs a soft contoured pillow to block light at the bottom edge of the mask (light most often seeps into sleep masks in this area). What is unique about the current invention's lower edge, light blocking pillow is the area which it spans, covering: the lower nose bridge; upper nasolabial area; cheekbones and then tapering off towards the lower temples and also the depth variations which are created by targeted darting.

Another differentiator in the current invention, is the darting and overall form of the lower edge pillow which create targeted, ergonomic, shaping that is the reverse shape of the hollows on the face and fills the void around the nose. The soft filling material used in the pillow has a good bounce to it and deforms to fit different face shapes without much force, and then softly fills in voids due to its resilience.

The way the current invention has been ergonomically designed is with reference to more than 280,534 data points resulting from 34 measurements collected, from 4,401 US women and 3,850 US men, across ages and ethnicities. Design driven by this data is a unique approach in this field (data collected using anthropometry software People Size 2003-4 and 2005-6). The result is a mask which is uniquely fitted. The sleep mask has been constructed to conform to the convex curvature of the face in such a way as that the outer surface of the mask is a longer line than the inner surface of the mask and the inner surface thus has full face contact.

The current invention deals with blink-space in a unique way, using the contoured light blocking pillow at the bottom of the mask to make a space behind the mask that angles back toward the forehead, creating a touch-free eye zone. The padding also tapers off toward the edge of the cheekbone, to keep the mask looking as slim and traditional as possible from the outside, while maintaining eye clearance and excellent light blocking. The method employed encourages adherence to the face and diminishes self-conscious feelings in a public venue by not having the bulky, “alien” look.

The approach taken to avoid squashing the ear is also distinct from prior art. The continuous, elastic securing band bypasses the ear area because of the angle of the securing band connectors. Then the way that it tethers to the single slider, so that the securing band originates from a single point, but then splits to the two-strap configuration in the area past the ears, ensures one will still get the hold benefit of increased strap surface area, without it running over the top of the ear.

The current invention employs a slip over continuous securing band, forming two straps being adjustable by securing band sliders and causes no discomfort, pulling or snagging issues when worn. The two elastic securing straps in the current invention also work in a unique way to allow for a high degree of adjustability. The single adjustment made with the sliders is distributed over the course of the whole elastic, allowing it to increase in length by an extra 50%. In other words, the adjustable range of increase is 0 to 50%, include increases such as 10%, 20%, 30% and 40%. This works particularly well on the high end of the sizing spectrum or for or any one with thick, full hair. Having a too-tight elastic squeezing your head for hours when you're trying to sleep can lead to migraines and a lot of general discomfort.

Light blocking effect can be a balance between shape and size. If you are trying to achieve a similar level of light-blocking, in a lightweight, elegant package, it is difficult, and that is what makes the current invention unique amongst prior art.

None of the prior art address light blocking or tension distribution with the precision that can be seen in the present subject. The therapeutic element of the current invention is far more subtle. The fabric which contacts the skin is a micro-encapsulated fiber, rich in antioxidant active principles Aloe Vera and Vitamin E. These capsules are microscopic, airtight and waterproof. They open to release the gel when the fabric is touched or rubbed. Every time the sleep mask is worn, a microfilm of vitamin E and aloe is applied to the skin. In addition, aloe is naturally anti-bacterial. In at least one preferred embodiment the outer portion of the mask is constructed from Mulberry Silk which is hypoallergenic.

The present invention also functions well in a seated or prone position.

Reference numerals are used throughout the drawings to show the various elements of the invention. The figures are intended for representative purposes only and should not be considered to be limiting in any respect.

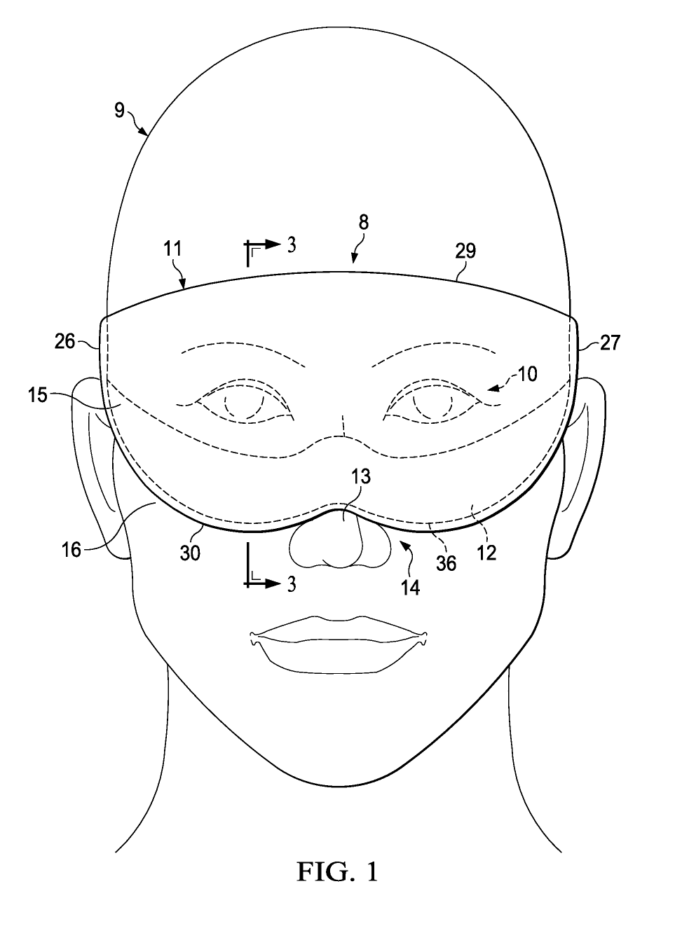

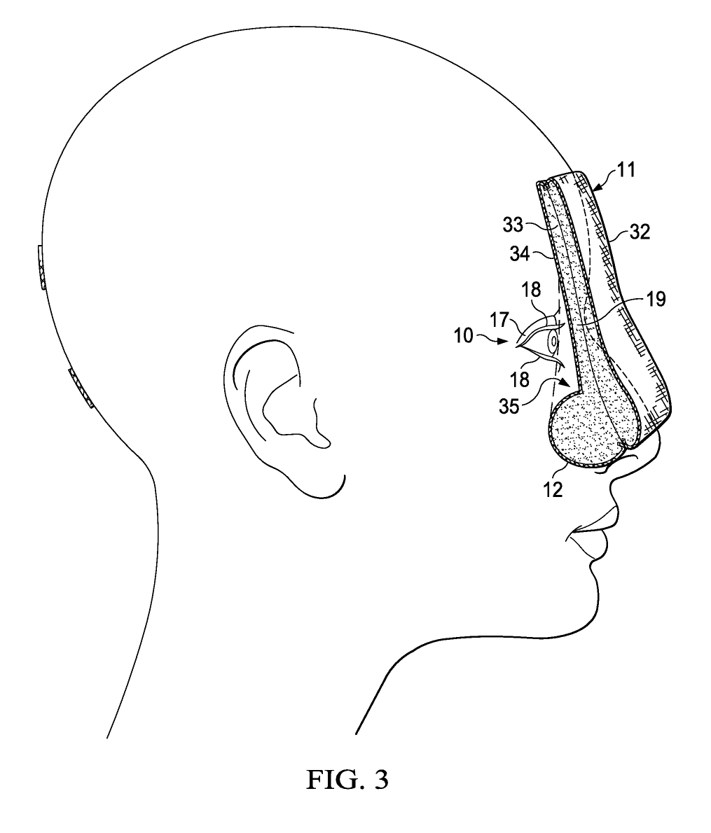

FIG. 1 illustrates a sleep mask 8 for a person 9. The forward portion 11 of the sleep mask is designed to cover the person's eyes 10. FIG. 3 is a cross-sectional detail which shows the four layers of material which form the forward or body portion and may comprise an opaque outer surface or outer layer 32; a layer of batting 33 and an inner liner or an inner layer or inner surface of fabric 34 which contacts the face. These 4 layers are held together by stitching 36 as indicated just inside the periphery. The inner liner 34 is preferably made from a cosmetic textile or cosmetotextile, which is any textile article containing a microencapsulated substance or preparation that is released over time, notably on human skin, and containing special functionalities such as cleansing, perfuming, changing appearance, etc. For example, inner liner 34 can be made from a microencapsulated aloe infused fabric and outer layer 32 of hypoallergenic silk but can be made of any material which is soft against the wearer's skin. The outer layer 32 of fabric may be Crepe de Chine fabric, for example, and the inner layer 34 of fabric may be a cosmetotextile lining. Additional layers of material could be added to both the outside surfaces of the mask 8 or on the inside of mask 8. Layers could be stitched to the outside surface with design or therapeutic properties or both. Similarly, the inside of mask 8 could be made of a plurality of inner thinner layers separated by fabric or separated simply by being different materials with different densities.

The outer layer 32 is defined by a first perimeter edge and the inner layer 34 is defined by a second perimeter edge. These perimeter edges are brought together and attached, by stitching for example, as can be seen in FIG. 3.

The batting 33, in the example above, may be a single or multiple layers. Batting 33 could be replaced with fill material like soft material, like cotton or polyester, or fill, like fiberfill or angel hair polyfill.

Depending on the size of sleep mask 8 the soft material, resilient enough to fill voids but flexible or pliable enough to deform to fit different face shapes can be added to the space between the inner layer 34 and outer layer 32 in amounts varying in a range of 3 to 5 grams, or more. Typically soft material, like cotton or polyester, or fill, like fiber fill or angel hair polyfill, is added in amounts like 3, 3.2, 3.4, 3.6, 3.8, 4.0, 4.2, 4.4, 4.6, 4.8 and 5 grams to the space between the inner layer 34 and outer layer 32.

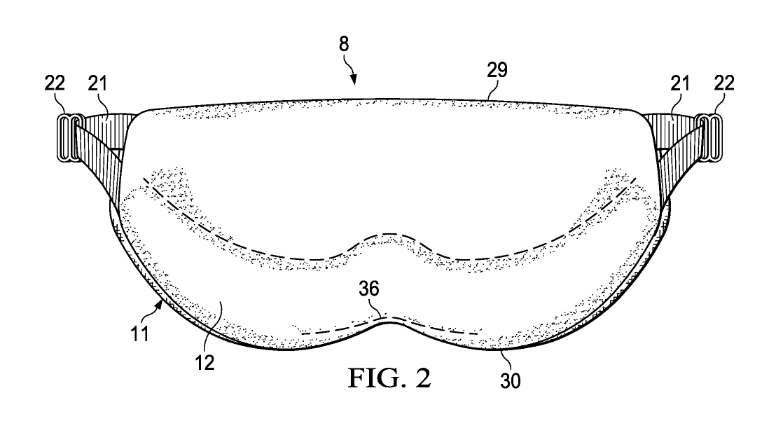

The forward portion includes a contoured light blocking pillow 12 at the bottom edge 30 of the mask 8 which conforms to the wearers face, following a line running over the lower nose bridge 13; upper nasolabial area 14; cheekbones 16 and then tapering off towards the lower temples 15. The light blocking pillow 12 is filled with material, resilient enough to fill voids but flexible or pliable enough to deform to fit different face shapes. The material can be fill, fiberfill or angel hair polyfill for example. Fiberfill is made up of individual fibers. FIG. 2 shows the pillow 12 form a rear elevation and illustrates the specific darting, folds or tucks sewn in to stitch 36, which is a darting stitch on the inner surface 34 which creates targeted, ergonomic, shaping. FIG. 8 further illustrates how the pillow 12 fills the voids around the nose and the hollows on the face from a bottom elevation. As shown in the figure, the pillow 12 directly contacts the face along its curved contours thereby effectively blocking out ambient light.

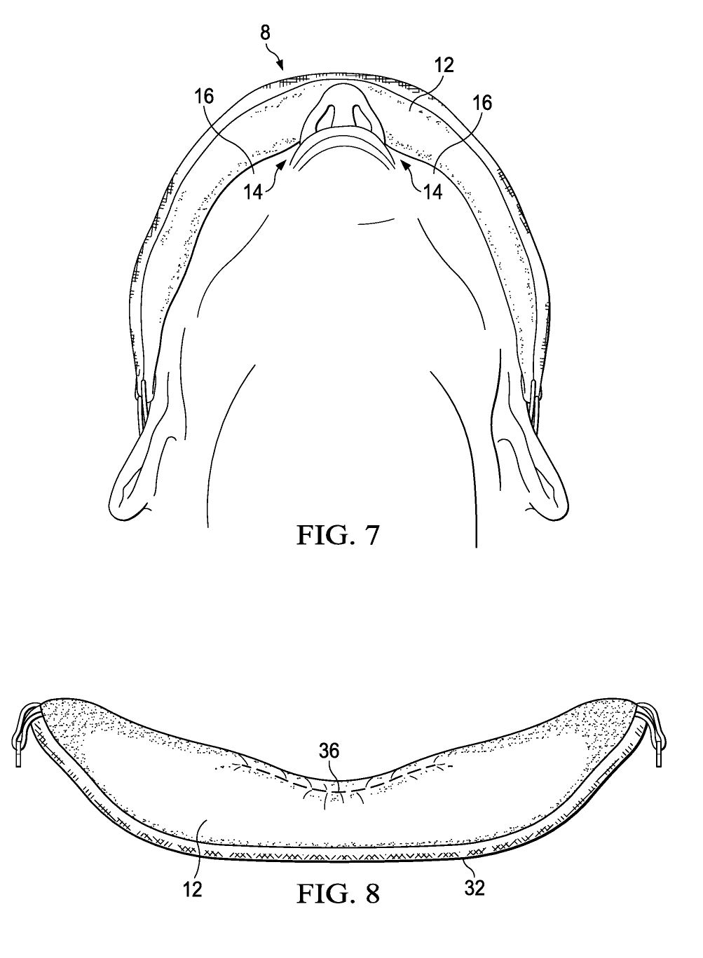

Light blocking pillow 12 is also filled material, resilient enough to fill voids but flexible or pliable enough to deform to fit different face shapes, such as fiberfill or batting. The pillow 12 can be formed on the lower 20% of the height of mask 8. In other words, the pillow 12 would be located on the inner side of the mask 8 against a wearer's face and would be formed along the entire lower 20% of the height of the mask 8 on the inside surface. In fact, the position of the pillow 12 on mask 8 can vary from the entire lower 15%, 16%, 17%, 18%, 19%, 21%, 22%, 23%, 24% and 25% of the height of the mask 8. The thickness of light blocking pillow 12 can vary along its length to provide for light blocking and at the same time direct facial contact along the contours of the face. For example, as shown in FIG. 7 the thickness of light blocking pillow 12 is at a maximum in the front of the face on both sides of the nose and tapers to a minimum along both sides of the face to left and right distal ends of sleep mask 8. The tapering of the thickness amount can be linear or more ergonomically, in other words the thickness can vary depending on the matching with the contours of the face.

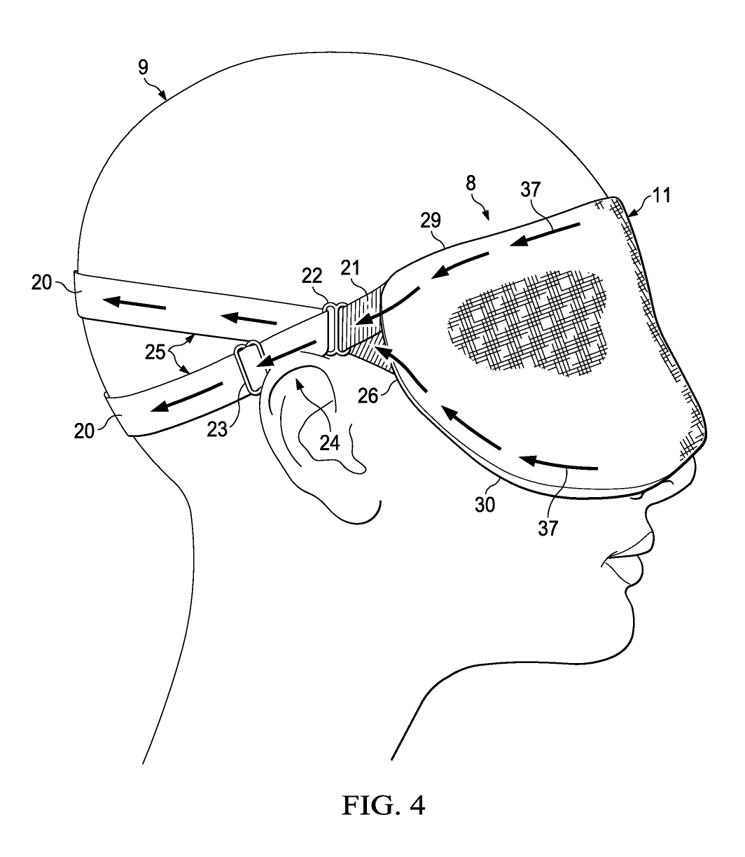

The light blocking pillow 12 also raises the mask 8 off the wearer's face 9 slightly, making a space 35 behind the mask 8 that angles back toward the forehead of the wearer, such that the person's eyelids 17 and eyelashes 18 do not contact the inner surface fabric when the sleep mask 8 is worn by the person 9, as shown in partial sectional detail in FIG. 4.

FIG. 8 shows the sleep mask 8 from a bottom perspective and highlights how the mask 8 has been constructed to curve to fit the face in such a way as that the outer surface 32 of the mask 8 is a longer line than the inner surface 34 of the mask 8 and the inner surface 34 thus has full face contact.

For example, the outer surface 32 has its longest length dimension measured between points where the securing bands 20 connect to each side of the outer surface 32. This length dimension can be 9.75 inches for example. In the same dimensional example, the height of mask 8 measured at the forward center of mask 8 from the top center of mask 8 to the center of the bottom edge specifically contoured for the nose is 4.0625 inches. At the same time the longest length of the inner surface 34 measured at the same points on the inner surface 34 is 9 inches. The range of length difference between the longest length of the outer surface 32 and the longest length of the inner surface 34 is in the range of 7 to 9%. The range includes the following percent values: 7.0 7.2, 7.4, 7.6, 7.8, 8.0, 8.2, 8.4, 8.6, 8.8 and 9.0

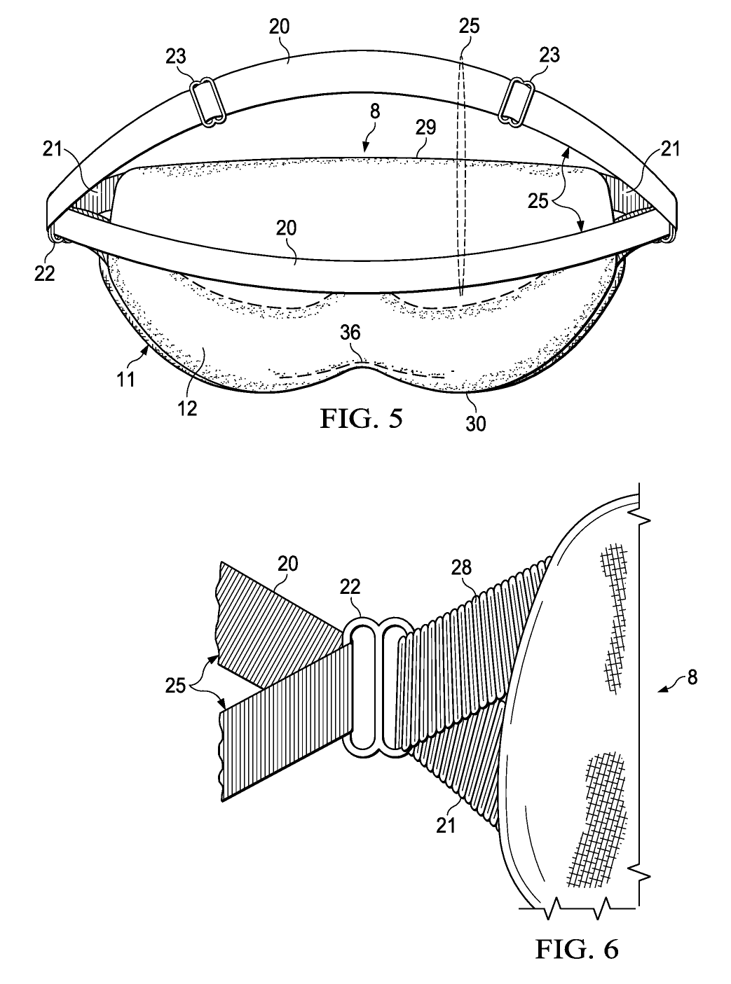

With reference to FIGS. 5, 6 & 7 a continuous, anti-tension elastic securing band 20 is attached to the forward portion of the mask 11 at the right and left edges 26 and 27 of the face portion. As such the sleep mask securing band 20 may be slipped over the person's head 9. The securing band may then be adjusted to achieve a secure fit, by two moving sliders 23 which alter the length of the securing band 20. Moving sliders are widely used in garments such as lingerie and need not be further described herein.

The securing band is tethered by a single slider 22 to securing band connectors 21 on the left and right edges 26 and 27 of the mask 8 which are angled so that the securing band 21 bypasses the ear area 24. The securing band originates from a single point at the securing band slider 22, but then splits into a two-strap configuration 25 in the area past the ears 24 increasing the surface area of the strap, maximizing hold, without running over the top of the ear 24. In the exemplary embodiment, the securing band 20 is formed from an elastic material and the securing band connectors 21 made from a micro adjustable corded ribbon. The securing band connectors are secured to the left and right edges 26, 27 of the mask 8 by stitching.

FIG. 5 illustrates the way tension is disbursed by interplay of the securing band connectors 21 and securing band 20. The securing band connectors 21 distribute tension by following the top and bottom lines of the mask 37, forming a ‘V’ shape. The continuous elastic securing band then forms two straps creating unbroken lines 37 from the connectors that continue to distribute tension from the top 29 and bottom 30 of the mask. The way the continuous band forms a two-strap configuration 25 lessens the tension on any one line, distributing it evenly over the two lines of elastic, to lessen the amount of pressure on the face and back of head, while creating a firm even tension on the face decreasing the chance of creating marks on the wearer's skin. The thickness of the facial batting 33 has been carefully adjusted to ensure facial pressure is minimized.

It is therefore submitted that the instant invention has been shown and described in what is considered to be the most practical and preferred embodiment. It is recognized, however, that departures may be made within the scope of the invention and that obvious modifications will occur. With respect to the above descriptions then, it is to be realized that the optimum dimensional relationships for the parts of the invention, to include variations in size, materials, shape, form, function, and manner of operation, assembly and use, are deemed readily apparent and obvious to one skilled in the art, and all equivalent relationships to those illustrated in the drawings and described in the specifications are intended to be encompassed by the present invention.

Therefore, the foregoing is considered as illustrative only of the principles of the invention. The invention has been shown in only one of its forms and is not thus limited but is susceptible to various changes and modifications without departing from the spirit thereof.

1. A light blocking, low volume sleep mask to be worn on a face to cover a horizontal space between left and right temples and a vertical space between a forehead and a nose of a wearer, the sleep mask comprising:
an outer layer of material, defined by a first perimeter edge, facing away from the wearer;
an inner layer of material, defined by a second perimeter edge, contacting the wearer and attached to the first perimeter edge of the outer layer of material at the second perimeter edge;
at least two separate layers of batting between the outer layer and the inner layer;
a contoured light blocking pillow formed by the inner layer to block 100% of an ambient light to the wearer formed on a lower 20% of a vertical height of the sleep mask; and
two anti-tension securing bands to secure the sleep mask to a head of the wearer connected to the outer layer and inner layer;
wherein the first perimeter edge of the outer layer is longer in maximum length than the second perimeter edge.

2. The sleep mask of claim 1, further comprising anti-tension securing band connectors attached to both the anti-tension securing bands and the outer and inner layers.

3. The sleep mask of claim 1, wherein the anti-tension securing band connectors form a “V” shape following top and bottom lines defining top and bottom surfaces of the sleep mask.

4. The sleep mask of claim 1, wherein the anti-tension securing bands form two tension distributing straps.

5. The sleep mask of claim 1, wherein the light blocking pillow is a contoured filled pillow that includes darting which shapes the contoured filled pillow of the sleep mask to create targeted shaping that is the reverse shape of hollows on the face so as to ensure direct contact between facial contours and the sleep mask.

6. The sleep mask of claim 1, wherein the inner layer includes darting around an area on the inner layer contacting the nose of the wearer which shapes the inner layer of the sleep mask to create targeted shaping so as to ensure direct contact between facial contours and the sleep mask.

7. The sleep mask of claim 5, wherein the light blocking pillow is filled with fiberfill, with batting between the inner layer and the outer layer in deeper areas contacting the nose and under eyes so that the light blocking pillow can directly contact the face and block 100% of ambient light.

8. The sleep mask of claim 1, further comprising a space above the light blocking pillow between the inner layer and the face of the wearer that angles back toward the forehead of the wearer.

9. The sleep mask of claim 2, wherein the securing band connectors are located to bypass ear areas of the wearer when in use based on an angle of the securing band connectors and a location of the connectors.

10. The sleep mask of claim 1, wherein the inner layer is formed of aloe infused fabric which has been combined with hypoallergenic, breathable mulberry silk.
